(19) United States
(12) Patent Application Publication
(10) Pub. No.: US 2023/0210387 A1
(43) Pub. Date: Jul. 6, 2023

(54) SPHYGMOMANOMETRY CUFF AND SPHYGMOMANOMETER

(71) Applicant: OMRON HEALTHCARE Co., Ltd., Muko-shi (JP)

(72) Inventors: Terumasa NAITO, Kyoto (JP); Yukiya SAWANOI, Kyoto (JP)

(73) Assignee: OMRON HEALTHCARE Co., Ltd., Muko-shi (JP)

(21) Appl. No.: 18/120,081

(22) Filed: Mar. 10, 2023

Related U.S. Application Data
(63) Continuation of application No. PCT/JP2021/038967, filed on Oct. 21, 2021.

(30) Foreign Application Priority Data
Nov. 4, 2020 (JP) 2020-184630

(51) Int. Cl. A61B 5/022

(52) U.S. Cl. CPC A61B 5/02233; A61B 5/02208

(57) ABSTRACT

A blood pressure measuring cuff of the present invention includes: an outer cloth that extends in a longitudinal direction in a band shape and surrounds a site to be measured; a pressing fluid bag that is provided to extend along the longitudinal direction on a side of the outer cloth facing the site to be measured and compresses the site to be measured; a sound acquisition fluid bag that is provided between the outer cloth and the pressing fluid bag in a thickness direction perpendicular to the outer cloth and acquires a sound from the site to be measured via the pressing fluid bag; a first fluid pipe connected to the pressing fluid bag so as to be capable of flowing a fluid; and a second fluid pipe connected to the sound acquisition fluid bag so as to be capable of flowing a fluid, separately from the first fluid pipe.

### CROSS-REFERENCE TO RELATED APPLICATION

This is a continuation application of International Application No. PCT/JP2021/038967, with an International filing date of Oct. 21, 2021, which claims priority of Japanese Patent Application No. 2020-184630 filed on Nov. 4, 2020, the entire content of which is hereby incorporated by reference.

### TECHNICAL FIELD

The present invention relates to a blood pressure measuring cuff, and more particularly to a blood pressure measuring cuff that compresses a site to be measured to acquire a Korotkoff sound. Furthermore, the present invention also relates to a sphygmomanometer including such a blood pressure measuring cuff to measure a blood pressure based on the Korotkoff sound.

### BACKGROUND ART

Conventionally, as this type of blood pressure measuring cuff, for example, as disclosed in Patent Literature 1 (JP S58-155841 A), a blood pressure measuring cuff including an ischemic cuff for compressing a site to be measured (an upper arm) and a sound collecting cuff disposed in a partial region of the ischemic cuff on a side facing the site to be measured is known. Similarly, as disclosed in Patent Literature 2 (JP 2012-61104 A), a blood pressure measuring cuff including an ischemic air bag for compressing a site to be measured (an upper arm) and first and second Korotkoff sound detecting air bags disposed in a partial region on a side facing the site to be measured in the ischemic air bag is known.

### SUMMARY OF INVENTION

However, in the blood pressure measuring cuffs of Patent Literatures 1 and 2 described above, since the sound collecting cuff (or first and second Korotkoff sound detecting air bags) is disposed only in a partial region of the ischemic cuff (or ischemic air bag) on the side facing the site to be measured, there is a problem that the sound collection is not stable when a position (in particular, a circumferential position) where the cuff is worn with respect to the site to be measured (upper arm) varies.

Therefore, an object of the present invention is to provide a blood pressure measuring cuff that acquires a Korotkoff sound by pressing a site to be measured, the blood pressure measuring cuff being capable of stably acquiring the Korotkoff sound. Furthermore, another object of the present invention is to provide a sphygmomanometer including such a blood pressure measuring cuff and capable of accurately measuring a blood pressure.

In order to solve the above-mentioned problem, a blood pressure measuring cuff that compresses a site to be measured to acquire a Korotkoff sound, the blood pressure measuring cuff of the present disclosure comprises:

an outer cloth extending in a longitudinal direction in a band shape and surrounding the site to be measured;

a pressing fluid bag that is provided to extend along the longitudinal direction on a side of the outer cloth facing the site to be measured, and compresses the site to be measured;

a sound acquisition fluid bag that is provided between the outer cloth and the pressing fluid bag in a thickness direction perpendicular to the outer cloth, and acquires a sound from the site to be measured via the pressing fluid bag;

a first fluid pipe connected to the pressing fluid bag so as to be capable of flowing a fluid; and

a second fluid pipe connected to the sound acquisition fluid bag so as to be capable of flowing a fluid, separately from the first fluid pipe.

In the present specification, the “site to be measured” includes an upper limb such as an upper arm and a wrist or a lower limb such as an ankle, and typically refers to a rod-like site.

The “side facing the site to be measured” means a side facing the site to be measured in a state where the blood pressure measuring cuff is worn around the site to be measured (This is referred to as a “worn state”).

In the blood pressure measuring cuff, the “longitudinal direction” means a direction in which the outer cloth extends in a band shape, and corresponds to a circumferential direction surrounding the site to be measured in the worn state. A “width direction” described later means a direction perpendicular to the longitudinal direction in a plane along the outer cloth, and corresponds to a direction in which an artery passes through the site to be measured in the worn state. Furthermore, the “thickness direction” means a direction perpendicular to both the longitudinal direction and the width direction (that is, the outer cloth), and corresponds to a direction perpendicular to an outer peripheral surface of the site to be measured in the worn state.

In another aspect, a sphygmomanometer that calculates a blood pressure by a Korotkoff sound generated by a site to be measured, the sphygmomanometer of the present disclosure comprises:

the blood pressure measuring cuff described above;

a pressure device that is connected to the first fluid pipe so as to be capable of flowing a fluid, and supplies a fluid to the pressing fluid bag through the first fluid pipe to pressurize the pressing fluid bag or discharges a fluid from the pressing fluid bag through the first fluid pipe to depressurize the pressing fluid bag;

a sound detection device that is connected to the second fluid pipe so as to be capable of flowing a fluid, and detects a sound from the sound acquisition fluid bag through the second fluid pipe;

a first fluid system including the pressing fluid bag and the first fluid pipe;

a second fluid system including the sound acquisition fluid bag and the second fluid pipe, the first fluid system and the second fluid system being maintained so as not to be capable of flowing a fluid each other; and a blood pressure calculation unit that opens and closes the atmosphere release valve as the pressure device pressurizes or depressurizes the pressing fluid bag, and calculates a blood pressure of the site to be measured based on an output of the sound detection device according to the sound from the sound acquisition fluid bag.

The “pressure device” typically includes a pump and a valve.

The “sound detection device” typically includes a microphone.

### BRIEF DESCRIPTION OF DRAWINGS

The present invention will become more fully understood from the detailed description given hereinbelow and the accompanying drawings which are given by way of illustration only, and thus are not limitative of the present invention, and wherein:

FIG. 1 is a diagram illustrating an appearance of a sphygmomanometer including a blood pressure measuring cuff according to an embodiment of the present invention.

FIG. 2 is a diagram illustrating a block configuration of the sphygmomanometer.

FIG. 3A is a diagram schematically illustrating a planar layout of a sound acquisition fluid bag and a pressing fluid bag included in the cuff in a state where the cuff is unfolded. FIG. 3(B) is a diagram schematically illustrating cross sections of the sound acquisition fluid bag and the pressing fluid bag in an exploded state.

FIG. 4A is a diagram schematically illustrating a mode in which the cuff is worn around an outer periphery of a left upper arm as a site to be measured. FIG. 4B is a diagram schematically illustrating a K sound signal (representing a Korotkoff sound) acquired using a sound detection device (microphone) through the sound acquisition fluid bag. FIG. 4(C) is a diagram schematically illustrating a pressure fluctuation component acquired by a pressure sensor through the pressing fluid bag.

FIG. 5 is a diagram illustrating a flow of blood pressure measurement by the sphygmomanometer.

FIG. 6A is a diagram illustrating a mode of setting a dimension in a longitudinal dimension and a dimension in a width direction of the sound acquisition fluid bag.

FIG. 6B is a diagram illustrating an amplitude of the K sound signal in a case where the dimension in the longitudinal direction of the sound acquisition fluid bag is set to various values. FIG. 6C is a diagram illustrating an amplitude of the K sound signal in a case where the dimension in the width direction of the sound acquisition fluid bag is set to various values.

FIG. 7A is a diagram illustrating a mode in which a position where the cuff is worn on the site to be measured, particularly a circumferential position of the sound acquisition fluid bag is changed in three ways. FIG. 7B is a diagram illustrating an amplitude of the K sound signal in a case where the circumferential position of the sound acquisition fluid bag of the cuff (example) is changed in three ways. FIG. 7C is a diagram illustrating an amplitude of the K sound signal in a case where a circumferential position of a sound acquisition fluid bag of a cuff (one in which the sound acquisition fluid bag is disposed under the pressing fluid bag) of Comparative Example 1 is changed in three ways.

FIG. 8A is a diagram illustrating a power spectrum of a sound acquired by the microphone in a case where the cuff (example) acquires a sound from the site to be measured (with K sound).

FIG. 8B is a diagram illustrating a power spectrum of a sound acquired by the microphone in a case where the cuff (example) does not acquire a sound from the site to be measured (no K sound).

FIG. 9A is a diagram illustrating a power spectrum of a sound acquired by the microphone in a case where a cuff (a cuff in which an air pipe of a pressing fluid bag and an air pipe of a sound acquisition fluid bag are common) of Comparative Example 2 acquires a sound from the site to be measured (with K sound).

FIG. 9B is a diagram illustrating a power spectrum of a sound acquired by the microphone in a case where the cuff of Comparative Example 2 does not acquire a sound from the site to be measured (no K sound).

FIG. 10 is a diagram illustrating background noise (sound pressure level) of a sound acquired by the microphone before and after opening and closing of an atmosphere release valve connected to the sound acquisition fluid bag so as to be capable of flowing a fluid during blood pressure measurement by the sphygmomanometer.

FIG. 11 is a diagram illustrating a cuff pressure and a K sound signal obtained according to the flow of blood pressure measurement by the sphygmomanometer in a case where the atmosphere release valve is closed before the start of pressurization by a pump after the cuff (example) is worn on the site to be measured.

FIG. 12 is a diagram illustrating a cuff pressure and a K sound signal obtained according to the flow of blood pressure measurement by the sphygmomanometer in a case where the atmosphere release valve is closed before the cuff (example) is worn on the site to be measured (Comparative Example 3).

FIG. 13A is a diagram schematically illustrating a planar layout of a sound acquisition fluid bag and a pressing fluid bag included in a cuff of Modification 1 in a state where the cuff is unfolded. FIG. 13B is a diagram schematically illustrating cross sections of the sound acquisition fluid bag and the pressing fluid bag in an exploded state.

FIG. 14A is a diagram schematically illustrating a planar layout of a sound acquisition fluid bag and a pressing fluid bag included in a cuff of Modification 2 in a state where the cuff is unfolded. FIG. 14B is a diagram schematically illustrating cross sections of the sound acquisition fluid bag and the pressing fluid bag in an exploded state.

FIG. 15A is a diagram schematically illustrating a planar layout of a sound acquisition fluid bag and a pressing fluid bag included in a cuff of Modification 3 in a state where the cuff is unfolded. FIG. 15B is a diagram schematically illustrating cross sections of the sound acquisition fluid bag and the pressing fluid bag in an exploded state.

### DESCRIPTION OF EMBODIMENTS

Hereinafter, embodiments of the present invention will be described in detail with reference to the drawings.

### (Schematic Configuration of Sphygmomanometer)

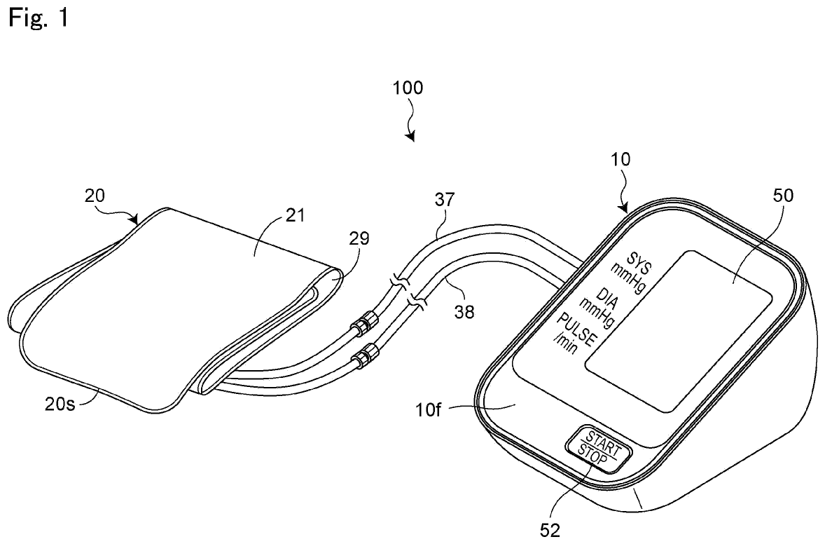

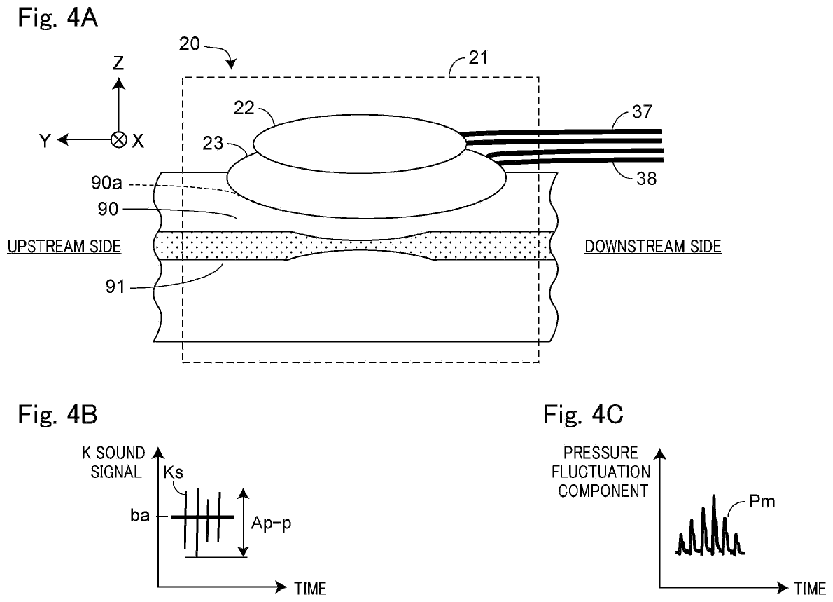

FIG. 1 illustrates an appearance of a sphygmomanometer 100 including a blood pressure measuring cuff 20 according to an embodiment of the present invention. The sphygmomanometer 100 roughly includes a cuff 20 worn around a rod-shaped site 90 to be measured (see FIG. 4A) such as an upper arm or a wrist, and a main body 10 connected to the cuff 20 so as to be capable of flowing a fluid through an air pipe 38 as a first fluid pipe and an air pipe 37 as a second fluid pipe.

### (Configuration of Blood Pressure Measuring Cuff)

As can be seen from FIG. 1, the cuff 20 is configured by making an outer cloth 21 having an elongated strip shape (in this example, a rounded rectangle) and an inner cloth 29 having a shape corresponding to the outer cloth 21 face each other, and sewing (or welding) peripheral edge parts 20s of the outer cloth 21 and the inner cloth 29.

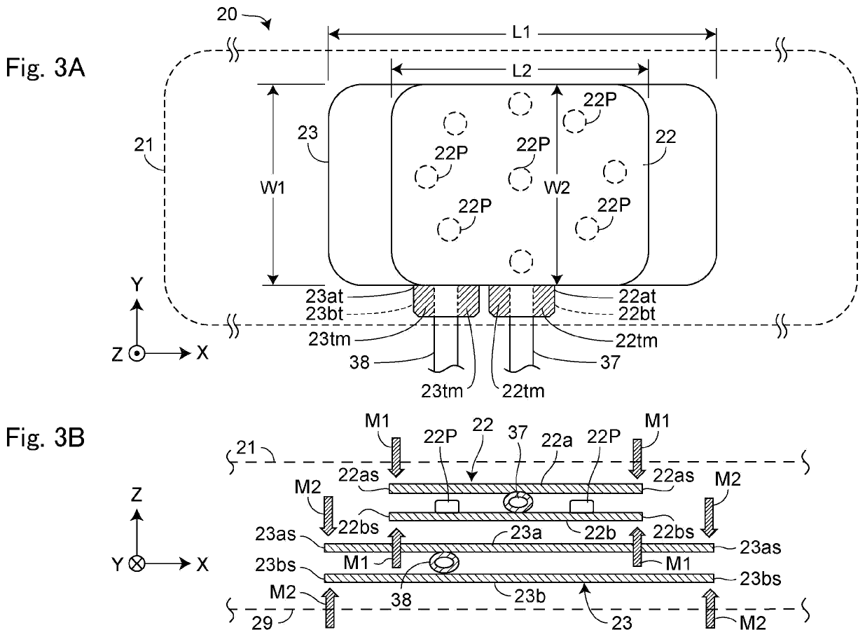

FIG. 3A schematically illustrates a planar layout of a sound acquisition fluid bag 22 and a pressing fluid bag 23 included in the cuff 20 in a state where the cuff 20 is unfolded. FIG. 3B schematically illustrates cross sections of the sound acquisition fluid bag 22 and the pressing fluid bag 23 in an exploded state. Here, regarding the cuff 20, a longitudinal direction X means a direction in which the outer cloth 21 extends in a band shape, and corresponds to a circumferential direction surrounding the site 90 to be measured in a worn state (see FIG. 4A). A width direction Y means a direction perpendicular to the longitudinal direction X in a plane along the outer cloth 21, and corresponds to a direction in which an artery 91 passes through the site 90 to be measured in the worn state. Furthermore, a thickness direction Z means a direction perpendicular to both the longitudinal direction X and the width direction Y (that is, outer cloth 21), and corresponds to a direction perpendicular to an outer peripheral surface of the site 90 to be measured in the worn state.

As can be seen from FIG. 3B, in this example, the cuff 20 includes the pressing fluid bag 23 and the sound acquisition fluid bag 22 configured separately from the pressing fluid bag 23 between the inner cloth 29 and the outer cloth 21. The pressing fluid bag 23 is provided on a side of the inner cloth 29 mainly to compress the site 90 to be measured. The sound acquisition fluid bag 22 is provided between the outer cloth 21 and the pressing fluid bag 23 in order to acquire a sound from the site 90 to be measured via the pressing fluid bag 23. In this example, the sound acquisition fluid bag 22 is partially bonded to the pressing fluid bag 23 so as not to be displaced with respect to the pressing fluid bag 23. The pressing fluid bag 23 is partially bonded to the outer cloth 21 so as not to be displaced with respect to the outer cloth 21.

As can be seen from FIG. 3A, the pressing fluid bag 23 has a substantially rectangular shape with rounded corners extending along the longitudinal direction X in a plane along the outer cloth 21. A dimension of the pressing fluid bag 23 in the longitudinal direction X is set to L1=235 mm, and a dimension of the pressing fluid bag 23 in the width direction Y is set to W1=125 mm. The sound acquisition fluid bag 22 has a substantially rectangular shape with round corners smaller than those of the pressing fluid bag 23 in a plane along the outer cloth 21. A dimension of the sound acquisition fluid bag 22 in the longitudinal direction X is set to L2=125 mm, and a dimension of the sound acquisition fluid bag 22 in the width direction Y is set to W2=125 mm. A specific method of setting the plane-direction dimensions L1, W1, L2, and W2 will be described later. In this example, a center of the pressing fluid bag 23 coincides with a center of the sound acquisition fluid bag 22.

As can be seen from FIG. 3B, the pressing fluid bag 23 includes a pair of sheets 23a and 23b facing each other in the thickness direction Z, and peripheral edge parts 23as and 23bs of the pair of sheets 23a and 23b are annularly joined (in this example, welded) to each other as indicated by arrows M2 to form a bag shape. The sound acquisition fluid bag 22 includes a pair of sheets 22a and 22b facing each other in the thickness direction Z, and peripheral edge parts 22as and 22bs of the pair of sheets 22a and 22b are annularly joined to each other as indicated by arrows M1 to form a bag shape. In this example, the sheets 23a, 23b, 22a, and 22b are made of polyurethane resin.

The pair of sheets 23a and 23b constituting the pressing fluid bag 23 has substantially rectangular tabs 23at and 23bt protruding in the width direction (−Y direction) in FIG. 3A at positions corresponding to each other. In a state where the air pipe 38 is sandwiched between the tabs 23at and 23bt, parts 23tm and 23tm (indicated by hatching) of the tabs 23at and 23bt corresponding to both sides of the air pipe 38 are entirely welded, so that the air pipe 38 is connected to the pressing fluid bag 23 so as to be capable of flowing a fluid. The pressing fluid bag 23 can be expanded by being supplied with air through the air pipe 38 and can be contracted by being discharged with air. Similarly, the pair of sheets 22a and 22b constituting the sound acquisition fluid bag 22 has substantially rectangular tabs 22at and 22bt protruding in the width direction (−Y direction) in FIG. 3A at positions corresponding to each other. The air pipe 37 is connected to the sound acquisition fluid bag 22 so as to be capable of flowing a fluid by entirely welding parts 22tm and 22tm (indicated by hatching) of the tabs 22at and 22bt corresponding to both sides of the air pipe 37 with the air pipe 37 interposed between the tabs 22at and 22bt. A sound acquired by the sound acquisition fluid bag 22 is transmitted to the main body 10 through the air pipe 37 (Details will be described later).

A plurality of protrusions 22p, 22p, . . . as spacers are provided in a gap between the pair of sheets 22a and 22b facing each other and forming the sound acquisition fluid bag 22. In this example, the protrusions 22p, 22p, . . . each have a short columnar shape, and are integrally formed with the sheet 22b disposed on a side of the pressing fluid bag 23. Thus, the spacers can be easily configured. In this example, these protrusions 22p, 22p, . . . are dispersedly arranged at substantially equal intervals in a plane (XY plane) along the outer cloth 21. This prevents the pair of sheets 22a and 22b from coming into close contact with each other during blood pressure measurement. Therefore, as described later, the sound acquisition fluid bag 22 can stably acquire a sound from the site 90 to be measured via the pressing fluid bag 23. As a result, the Korotkoff sound can be stably acquired.

The outer cloth 21 can be curved or bent, but is configured not to substantially expand and contract in order to restrict the entire expansion of the sound acquisition fluid bag 22 and the pressing fluid bag 23 in a direction away from the site 90 to be measured at the time of blood pressure measurement. On the other hand, the inner cloth 29 can be curved or is bendable, and is easily stretchable so that the pressing fluid bag 23 easily compresses the site 90 to be measured during blood pressure measurement. Here, the outer cloth 21 and the inner cloth 29 are not limited to those knitted, and may be made of one layer or a plurality of layers of resin. Dimensions of the outer cloth 21 and the inner cloth 29 in the longitudinal direction X are set to be longer than a peripheral length of the site 90 to be measured (in this example, an upper arm). Dimensions of the outer cloth 21 and the inner cloth 29 in the width direction Y are set to be slightly larger than dimensions of the pressing fluid bag 23 (and the sound acquisition fluid bag 22) in the width direction Y Note that the inner cloth 29 is provided to protect the sound acquisition fluid bag 22 and the pressing fluid bag 23, and can be omitted for blood pressure measurement.

### (Configuration of Main Body)

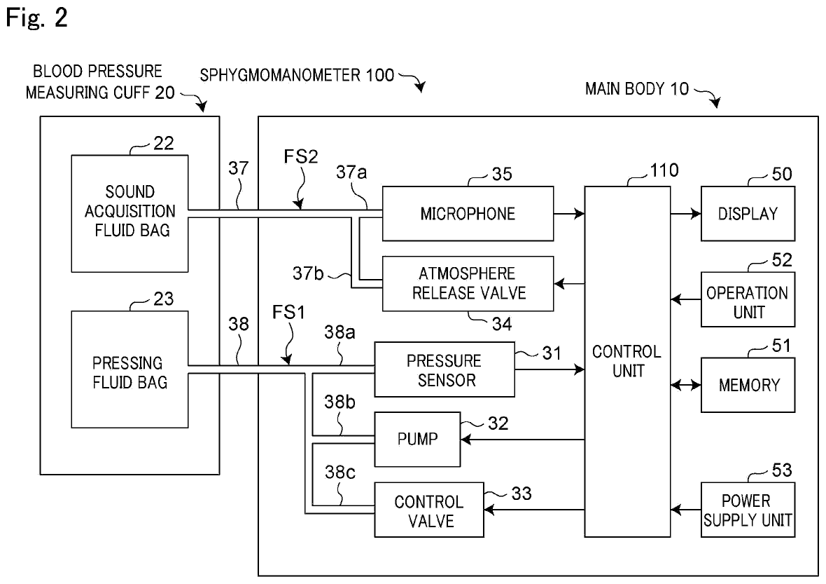

As illustrated in FIG. 2, the main body 10 includes a control unit 110, a display 50, an operation unit 52, a memory 51 as a storage unit, a power supply unit 53, a pressure sensor 31, a pump 32 and a control valve 33 as a pressure device, a microphone 35 as a sound detection device, and an atmosphere release valve 34. In this example, an air pipe 38a connected to the pressure sensor 31, an air pipe 38b connected to the pump 32, and an air pipe 38c connected to the control valve 33 join to form one air pipe 38 connected to the pressing fluid bag 23 so as to be capable of flowing a fluid. The air pipe 38 as the first fluid pipe is a generic term including these air pipes 38a, 38b, and 38c. Furthermore, an air pipe 37a connected to the microphone 35 and an air pipe 37b connected to the atmosphere release valve 34 join to form one air pipe 37 connected to the sound acquisition fluid bag 22 so as to be capable of flowing a fluid. The air pipe 37 as the second fluid pipe is a generic term including these air pipes 37a and 37b.

As illustrated in FIG. 1, the display 50 and the operation unit 52 are disposed on a front panel 10f of the main body 10. In this example, the display 50 includes a liquid crystal display (LCD), and displays predetermined information in accordance with a control signal from the control unit 110. In this example, a systolic blood pressure (SYS, units; mmHg), a diastolic blood pressure (DIA, units; mmHg), and a pulse rate PULSE (unit; beat/min) are displayed. Note that the display 50 may be an organic electro luminescence (EL) display, or may include a light emitting diode (LED).

In this example, the operation unit 52 includes a measurement switch (for simplicity, denoted by the same reference sign 52) for receiving an instruction to start/stop the measurement of a blood pressure, and inputs an operation signal according to an instruction of the user to the control unit 110. Specifically, when the measurement switch 52 is pressed, an operation signal indicating that blood pressure measurement should be started is input to the control unit 110, and the control unit 110 starts blood pressure measurement described later (When the blood pressure measurement is completed, the operation is automatically stopped). When the measurement switch 52 is pressed during the execution of the blood pressure measurement, the control unit 110 urgently stops the blood pressure measurement.

The memory 51 illustrated in FIG. 2 stores data of a program for controlling the sphygmomanometer 100, setting data for setting various functions of the sphygmomanometer 100, data of a measurement result of a blood pressure value, and the like. Furthermore, the memory 51 is used as a work memory or the like when the program is executed.

The control unit 110 includes a central processing unit (CPU) and controls the entire operation of the sphygmomanometer 100. Specifically, the control unit 110 acts as a pressure control unit according to a program for controlling the sphygmomanometer 100 stored in the memory 51, and performs control to drive the pump 32 and the control valve 33 as a pressure device according to an operation signal from the operation unit 52. Furthermore, the control unit 110 works as a blood pressure calculation unit, calculates a blood pressure value based on an output of the microphone 35, and controls the display 50 and the memory 51. A specific method of the blood pressure measurement will be described later.

The pressure sensor 31 is a piezoresistive pressure sensor in this example, and outputs the pressure (This is referred to as “cuff pressure Pc”) of the pressing fluid bag 23 contained in the cuff 20 as an electric resistance due to the piezoresistive effect through the air pipe 38. In this example, the control unit 110 includes an oscillation circuit that oscillates at an oscillation frequency according to the electric resistance from the pressure sensor 31, and obtains the cuff pressure Pc according to the oscillation frequency.

The pump 32 supplies air to the pressing fluid bag 23 included in the cuff 20 through the air pipe 38 based on a control signal given from the control unit 110. As a result, the pressure (cuff pressure Pc) of the pressing fluid bag 23 is pressurized.

The control valve 33 is a normally-open type electromagnetic control valve, and is opened and closed to control the cuff pressure by discharging or enclosing the air in the pressing fluid bag 23 through the air pipe 38 based on the control signal given from the control unit 110.

The microphone 35 detects a sound acquired by the sound acquisition fluid bag 22 through the air pipe 37, and outputs an electric signal according to the sound to the control unit 110. In this example, the control unit 110 performs filtering including fast Fourier transform (FFT) from an electric signal output from the microphone 35 to extract a K sound signal (represented by Ks) representing a Korotkoff sound. As illustrated in FIG. 4B, the K sound signal Ks is typically obtained as a pulse-like signal oscillating at a high level and a low level with respect to a reference level ba. In FIG. 4B, an amplitude of a peak-to-peak of the K sound signal Ks is represented by Ap-p.

The atmosphere release valve 34 illustrated in FIG. 2 is a normally-open type electromagnetic control valve, and is opened and closed to open or seal a second fluid system FS2 including the sound acquisition fluid bag 22 and the air pipe 37 to the atmosphere based on a control signal given from the control unit 110.

In this example, a first fluid system FS1 including the pressing fluid bag 23, the air pipe 38, the pressure sensor 31, the pump 32, and the control valve 33, and the second fluid system FS2 including the sound acquisition fluid bag 22, the air pipe 37, the microphone 35, and the atmosphere release valve 34 are separated from each other so as not to be capable of flowing a fluid, and the separation is maintained also in the main body 10. As a result, it is possible to prevent the pulse sound (pulse wave sound) from being mixed from the first fluid system FS1 with respect to the sound (including a Korotkoff sound component) passing through the second fluid system FS2 (in particular, the air pipe 37). Therefore, the Korotkoff sound can be more stably acquired (Details will be described later).

The power supply unit 53 supplies power to the control unit 110, the display 50, the memory 51, the pressure sensor 31, the pump 32, the control valve 33, the microphone 35, and the atmosphere release valve 34.

### (Mode of Wearing Blood Pressure Measuring Cuff)

As illustrated in FIG. 4A (a cross section along the artery 91 passing through the site 90 to be measured), the cuff 20 is worn in a mode in which the longitudinal direction X of the cuff 20 surrounds the outer peripheral surface of the site 90 to be measured (in this example, the left upper arm). At the time of wearing, the outer cloth 21 is fixed by a hook-and-loop fastener (not illustrated) so as not to be loosened. Note that, in FIG. 4A, the inner cloth 29 is not illustrated for simplicity, and the pressing fluid bag 23 and the sound acquisition fluid bag 22 are each drawn in an elliptical shape. In this worn state, the inner cloth 29 not illustrated in the drawing, the pressing fluid bag 23, the sound acquisition fluid bag 22, and the outer cloth 21 are arranged in this order in the thickness direction Z with respect to the outer peripheral surface of the site 90 to be measured. Note that, in the worn state, since the air pipes 37 and 38 extend toward a downstream side (−Y direction) of a blood flow passing through the artery 91, the air pipes 37 and 38 do not interfere with the wearing.

### (Blood Pressure Measurement)

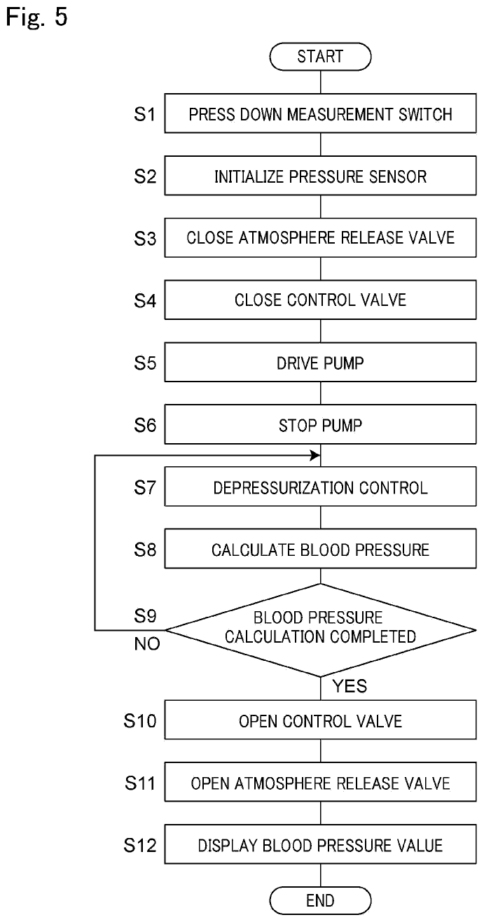

FIG. 5 illustrates an operation flow when a user performs blood pressure measurement with the sphygmomanometer 100.

When the user instructs to start the measurement using the measurement switch 52 provided on the main body 10 in the worn state where the cuff 20 is worn on the site 90 to be measured (step S1 in FIG. 5), the control unit 110 performs initialization (step S2 in FIG. 5). Specifically, the control unit 110 initializes a processing memory area and stops the pump 32, and performs 0 mmHg adjustment (The atmospheric pressure is set to 0 mmHg) of the pressure sensor 31 in a state where the control valve 33 is opened. At this time, the atmosphere release valve 34 is in an open state.

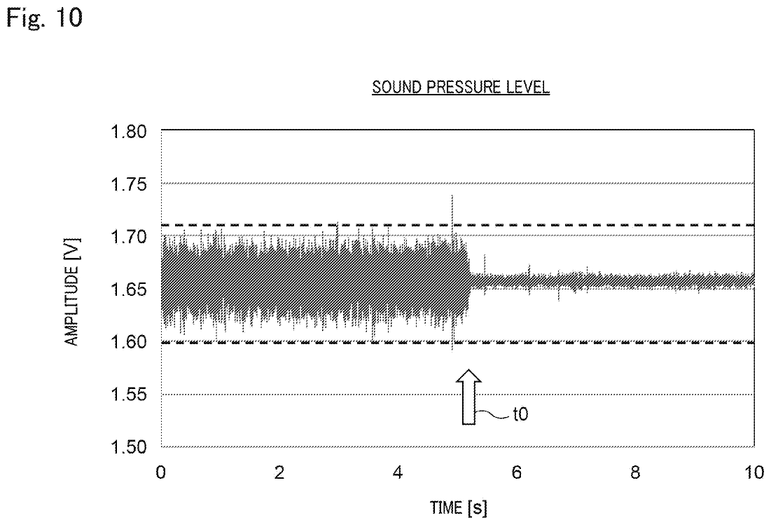

Next, the control unit 110 closes the atmosphere release valve 34 (step S3). The reason why the atmosphere release valve 34 is closed at this stage after the cuff 20 is worn on the site 90 to be measured and before the pressurization of the pressing fluid bag 23 is started is to seal an appropriate amount of air in the sound acquisition fluid bag 22 in order to acquire the Korotkoff sound from the site 90 to be measured via the pressing fluid bag 23. Note that FIG. 10 illustrates background noise (sound pressure level) of a sound acquired by the microphone 35 before and after time t0 when the atmosphere release valve 34 is closed at time t0 during the blood pressure measurement by the sphygmomanometer 100. As can be seen from FIG. 10, closing the atmosphere release valve 34 contributes to improving a signal-to-noise ratio (S/N ratio) in acquiring the Korotkoff sound since it reduces background noise.

Subsequently, the control unit 110 acts as a pressure control unit, closes the control valve 33 (step S4), drives the pump 32, and starts pressurizing the cuff 20 (step S5). That is, the control unit 110 supplies air from the pump 32 to (the pressing fluid bag 23 included in) the cuff 20 through the air pipe 38. At the same time, the pressure sensor 31 acts as a pressure detection unit to detect the pressure of the pressing fluid bag 23 through the air pipe 38. The control unit 110 controls a pressurization rate by the pump 32 based on an output of the pressure sensor 31.

At this time, expansion of the pressing fluid bag 23 illustrated in FIG. 4A in a direction away from the site 90 to be measured together with the sound acquisition fluid bag 22 is regulated by the outer cloth 21 as a whole. Therefore, the pressing fluid bag 23 expands in a direction of pressing a facing region 90A of the site 90 to be measured. As a result, the region 90A of the site 90 to be measured facing the pressing fluid bag 23 is pressed, and the artery 91 passing through the region 90A is ischemic.

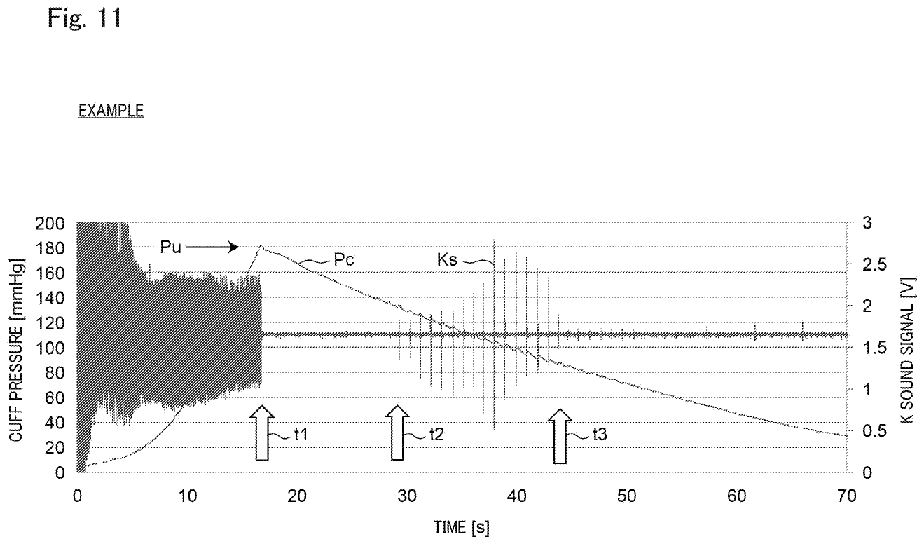

Next, the control unit 110 determines whether or not the pressure (cuff pressure Pc) of the cuff 20 (in this example, the pressing fluid bag 23) has reached a predetermined value Pu (for example, illustrated in FIG. 11) on the basis of an output of the pressure sensor 31. Here, the value Pu may be set to be, for example, 280 mmHg so as to sufficiently exceed an assumed blood pressure value of the subject, or may be set to be a blood pressure value of the subject measured last time plus 40 mmHg. In this example, as illustrated in FIG. 11, it is assumed that Pu=180 mmHg is set in advance. The control unit 110 continues pressurization until the cuff pressure Pc reaches the above-described value Pu=180 mmHg, and stops the pump 32 when the cuff pressure Pc reaches the above-described value Pu (step S6). In the example of FIG. 11, the cuff pressure Pc reaches the above-described value Pu at time t1, and the pump 32 is stopped.

Subsequently, the control unit 110 gradually opens the control valve 33 (step S7 in FIG. 5). As a result, the cuff pressure Pc is reduced at a substantially constant speed. In this example, in this depressurization process, the sound acquisition fluid bag 22 acquires a sound from the site 90 to be measured via the pressing fluid bag 23. Moreover, the microphone 35 detects the sound acquired by the sound acquisition fluid bag 22 through the air pipe 37. The microphone 35 outputs an electric signal according to the sound to the control unit 110. The control unit 110 performs filtering including fast Fourier transform (FFT) from the electric signal output from the microphone 35 to extract a K sound signal Ks representing a Korotkoff sound. In the example of FIG. 11, the K sound signal Ks starts to be observed at time t2, gradually increases to indicate a maximum value, then gradually decreases, and disappears at time t3.

The control unit 110 acts as a blood pressure calculation unit, and attempts to calculate a blood pressure value (systolic blood pressure (SYS) and diastolic blood pressure (DIA)) based on the K sound signal Ks acquired at this time (step S8 in FIG. 5). In the example of FIG. 11, the cuff pressure Pc detected by the pressure sensor 31 at time t2 is calculated as the systolic blood pressure SYS. Furthermore, the cuff pressure Pc detected by the pressure sensor 31 at time t3 is calculated as the diastolic blood pressure DIA.

Furthermore, a pulse wave signal (pressure fluctuation component) Pm (illustrated in FIG. 4C) as pulse wave information by a pulse wave is superimposed on the cuff pressure Pc detected by the pressure sensor 31 from the pressing fluid bag 23 through the air pipe 38. In this example, the control unit 110 calculates a pulse rate PULSE (beats/min) on the basis of the pulse wave signal Pm.

In a case where the blood pressure value and the pulse rate cannot be calculated yet due to lack of data (NO in step S9 in FIG. 5), the control unit 110 repeats the processing of steps S7 to S9 until the blood pressure value and the pulse rate can be calculated.

When the blood pressure value and the pulse rate can be calculated in this manner (Yes in step S9), the control unit 110 acts as a pressure control unit, opens the control valve 33, and performs control to rapidly exhaust the air in the cuff 20 (pressing fluid bag 23) (step S10). Furthermore, the atmosphere release valve 34 is opened (step S11).

Thereafter, the control unit 110 displays the calculated blood pressure value and pulse rate on the display 50 (step S12), and performs control to store the blood pressure value and the pulse rate in the memory 51.

In this manner, in the sphygmomanometer 100 including the cuff 20, the sound acquisition fluid bag 22 acquires the sound from the site 90 to be measured via the pressing fluid bag 23. In the worn state, the pressing fluid bag 23 extends along the circumferential direction of the site 90 to be measured. Therefore, even if a position (in particular, a circumferential position) where the cuff 20 (pressing fluid bag 23) is worn with respect to the site 90 to be measured varies, the influence on a level of the sound entering the pressing fluid bag 23 from the artery 91 passing through the site 90 to be measured is smaller than that in the conventional example, and as a result, the sound collection by the sound acquisition fluid bag 22 is stabilized. Therefore, the K sound signal Ks representing the Korotkoff sound can be stably acquired. As a result, the blood pressure can be accurately measured.

Note that, in the above example, the blood pressure value and the pulse rate are calculated in the depressurization process of the cuff 20 (pressing fluid bag 23), but the present invention is not limited thereto, and the blood pressure value and the pulse rate may be calculated in the pressurization process of the cuff 20 (pressing fluid bag 23).

### (Setting of Plane-Direction Dimensions of Pressing Fluid Bag and Sound Acquisition Fluid Bag)

The plane-direction dimensions of the pressing fluid bag 23 and the sound acquisition fluid bag 22 are set according to a cuff size (is set as a specification of the cuff, and defines plane-direction dimensions of the outer cloth 21 and the inner cloth 29). Generally, as the cuff size, XL (extra large), L (large), M (medium), and S (small) are set for the upper arm as illustrated in a “cuff size” field in Table 1 below. Furthermore, a wrist size is set.

TABLE 1

| | |
|---|---|
| Pressing fluid bag | Sound acquisition fluid bag |

| | L1 | W1 | L2 | W2 |
|---|---|---|---|---|
| Cuff size | [mm] | [mm] | [mm] | [mm] |
| XL | 380.0 | 180.0 | 95-380 | 90-180 |
| L | 312.5 | 150.0 | 78.1-312.5 | 75-150 |
| M | 235.0 | 125.0 | 58.8-235 | 62.5-125 |
| S | 167.0 | 90.0 | 41.8-167 | 45-90 |
| For wrist | 140 | 60 | 35-140 | 30-60 |

The dimension L1 in the longitudinal direction X and the dimension W1 in the width direction Y of the pressing fluid bag 23 are set to various values as shown in a “pressing fluid bag” column of Table 1 according to the cuff size corresponding to an arm circumference of the subject. That is, in the case of the cuff size XL (extra large) for the upper arm, the dimension L1 in the longitudinal direction X is set to 380.0 mm, and the dimension W1 in the width direction Y is set to 180.0 mm. For the cuff size L (large) for the upper arm, the dimension L1 in the longitudinal direction X is set to 312.5 mm, and the dimension W1 in the width direction Y is set to 150.0 mm. For the cuff size M (middle) for the upper arm, the dimension L1 in the longitudinal direction X is set to 235.0 mm, and the dimension W1 in the width direction Y is set to 125.0 mm. For the cuff size S (small) for the upper arm, the dimension L1 in the longitudinal direction X is set to 167.0 mm, and the dimension W1 in the width direction Y is set to 90.0 mm. For the wrist, the dimension L1 in the longitudinal direction X is set to 140 mm, and the dimension W1 in the width direction Y is set to 60 mm. Due to the setting of the plane-direction dimensions L1 and W1 of the pressing fluid bag 23, the cuff 20 can be fitted and worn to subjects of various arm circumferences and wrist circumferences.

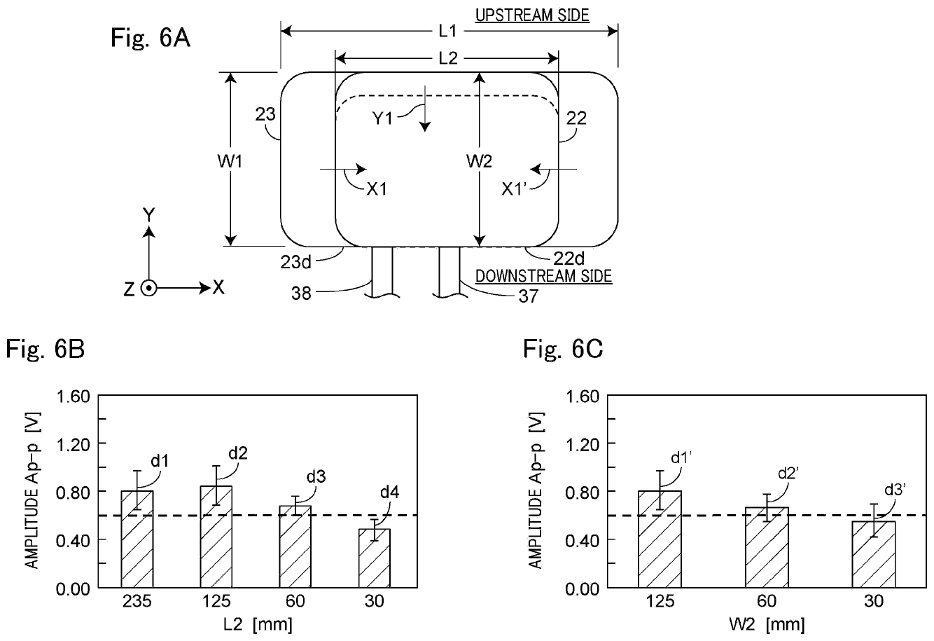

FIG. 6A illustrates a mode in which a dimension L2 in the longitudinal direction X and a dimension W2 in the width direction Y of the sound acquisition fluid bag 22 are set. For example, when the dimension L2 of the sound acquisition fluid bag 22 in the longitudinal direction X is reduced from the maximum value, the dimension L2 is reduced while the center of the sound acquisition fluid bag 22 in the longitudinal direction X coincides with the center of the pressing fluid bag 23 in the longitudinal direction X as indicated by arrows X1 and X1′. For example, when the dimension W2 of the sound acquisition fluid bag 22 in the width direction Y is reduced from the maximum value, the dimension W2 is reduced while a side 22d on the downstream side of the sound acquisition fluid bag 22 coincides with a side 23d on the downstream side of the pressing fluid bag 23 as indicated by an arrow Y1. The reason is to prevent a pulse sound (pulse wave sound) from the upstream side of the artery 91 from being mixed into the sound acquisition fluid bag 22 as much as possible. In this example, the dimension L2 in the longitudinal direction X and the dimension W2 in the width direction Y of the sound acquisition fluid bag 22 are set to various values as illustrated in a “sound acquisition fluid bag” column of Table 1 according to the cuff size corresponding to the arm circumference of the subject. That is, in the case of the cuff size XL for the upper arm, the dimension L2 of the sound acquisition fluid bag 22 in the longitudinal direction X is set within a range of 95 mm to 380 mm, and accordingly, the dimension W2 of the sound acquisition fluid bag 22 in the width direction Y is set within a range of 90 mm to 180 mm. In the case of the cuff size L for the upper arm, the dimension L2 of the sound acquisition fluid bag 22 in the longitudinal direction X is set within a range of 78.1 mm to 312.5 mm, and accordingly, the dimension W2 of the sound acquisition fluid bag 22 in the width direction Y is set within a range of 75 mm to 150 mm. In the case of the cuff size M for the upper arm, the dimension L2 of the sound acquisition fluid bag 22 in the longitudinal direction X is set within a range of 58.8 mm to 235 mm, and accordingly, the dimension W2 of the sound acquisition fluid bag 22 in the width direction Y is set within a range of 62.5 mm to 125 mm. In the case of the cuff size S for the upper arm, the dimension L2 of the sound acquisition fluid bag 22 in the longitudinal direction X is set within a range of 41.8 mm to 167 mm, and accordingly, the dimension W2 of the sound acquisition fluid bag 22 in the width direction Y is set within a range of 45 mm to 90 mm. In the case of the wrist size, the dimension of the sound acquisition fluid bag 22 in the longitudinal direction X is set to a value within a range of 35 mm to 140 mm for the dimension L2, and accordingly, the dimension W2 of the sound acquisition fluid bag 22 in the width direction Y is set to a value within a range of 30 mm to 60 mm.

FIGS. 6B and 6C respectively illustrate an amplitude Ap-p (unit; volt) of a peak-to-peak of the K sound signal Ks in a case where the dimension L2 in the longitudinal direction X and the dimension W2 in the width direction Y of the sound acquisition fluid bag 22 are set to various values. Note that, for the pressing fluid bag 23, the dimension in the longitudinal direction X is fixed to L1=235.0 mm, and the dimension W1 in the width direction Y was fixed to 125.0 mm (These values correspond to setting values for the upper arm and the cuff size M). As can be seen from FIG. 6B, with respect to the dimension L2 in the longitudinal direction X of the sound acquisition fluid bag 22 under the condition of the dimension W2=125 mm in the width direction Y, an average value of the amplitudes Ap-p when L2=235 mm is about 0.80 volts, an average value of the amplitudes Ap-p when L2=125 mm is about 0.85 volts, an average value of the amplitudes Ap-p when L2=60 mm is about 0.68 volts, and an average value of the amplitudes Ap-p when L2=30 mm is about 0.48 volts. Note that d1, d2, d3, and d4 indicate a variation range of the amplitudes Ap-p at each L2 setting value in this case. As a result, with respect to the dimension L2 of the sound acquisition fluid bag 22 in the longitudinal direction X, when L2=125 mm corresponding to ½ of the dimension L1 (=235 mm) of the pressing fluid bag 23 in the longitudinal direction X, the amplitude Ap-p shows a maximum value, which has been found to be advantageous. Furthermore, under the condition of the dimension L2=235 mm in the longitudinal direction X, with respect to the dimension W2 of the sound acquisition fluid bag 22 in the width direction Y, an average value of the amplitudes Ap-p when W2=125 mm is about 0.80 volts, an average value of the amplitudes Ap-p when W2=60 mm is about 0.68 volts, and an average value of the amplitudes Ap-p when W2=30 mm is about 0.56 volts. Note that d1′, d2′, and d3′ indicate a variation range of the amplitude Ap-p at each W2 setting value in this case. As a result, when W2=125 mm corresponding to the dimension W1 (=125 mm) of the pressing fluid bag 23 in the width direction Y, the amplitude Ap-p shows a maximum value, which has been found to be advantageous. The reason why it is advantageous to set L2=125 mm and W2=125 mm in this manner is considered that in a case where the pair of sheets 22a and 22b constituting the sound acquisition fluid bag 22 is made of, for example, a general polyurethane resin, a natural frequency of the sound acquisition fluid bag 22 is substantially the same order with respect to the main frequency component of the Korotkoff sound. As a result, the sound acquisition fluid bag 22 can efficiently acquire the Korotkoff sound component from the site 90 to be measured.

In verification experiments 1 to 3 described below, for the cuff 20, as described above, the dimension of the pressing fluid bag 23 in the longitudinal direction X was set to L1=235 mm, and the dimension of the pressing fluid bag 23 in the width direction Y was set to W1=125 mm. The dimension of the sound acquisition fluid bag 22 in the longitudinal direction X was set to L2=125 mm, and the dimension of the sound acquisition fluid bag 22 in the width direction Y was set to W2=125 mm.

### (Verification Experiment 1)

In order to verify the effect that the Korotkoff sound can be stably acquired even if a position (in particular, a circumferential position) where the cuff 20 is worn on the site 90 to be measured varies due to the configuration in which the sound acquisition fluid bag 22 is disposed on the pressing fluid bag 23 (see FIG. 4A), the inventors of the present invention conducted the following verification experiment.

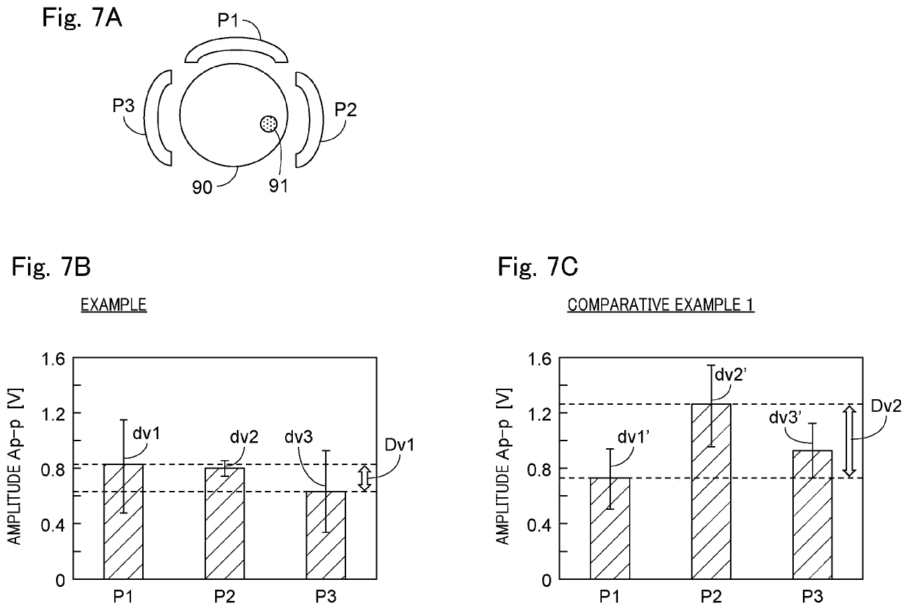

FIG. 7A illustrates a mode in which a position where the cuff 20 is worn on the site 90 to be measured, particularly, the circumferential position of the sound acquisition fluid bag 22 is changed in three ways as indicated by P1, P2, and P3. FIG. 7A corresponds to a cross section when the left upper arm as the site 90 to be measured is viewed from the upstream side of the artery 91. The circumferential position P2 corresponds to a position where the center of the sound acquisition fluid bag 22 faces the artery 91. The circumferential position P3 corresponds to a position opposite to the circumferential position P2 with respect to the site 90 to be measured. The circumferential position P1 corresponds to an intermediate position between the circumferential position P2 and the circumferential position P3 around the site 90 to be measured. FIG. 7B illustrates an amplitude Ap-p (unit; volt) of a peak-to-peak of the K sound signal Ks in a case where the circumferential position of the sound acquisition fluid bag 22 of the cuff 20 is changed in three ways as indicated by P1, P2, and P3. In this case, an average value of the amplitudes Ap-p at the circumferential position P1 was about 0.82 volts, an average value of the amplitudes Ap-p at the circumferential position P2 was about 0.80 volts, and an average value of the amplitudes Ap-p at the circumferential position P3 was about 0.64 volts. As a result, a change amount (maximum difference) Dv1 of the average value of the amplitudes Ap-p due to the change of the circumferential positions P1, P2, and P3 was about 0.18 volt. Note that dv1, dv2, and dv3 indicate a variation range of the amplitude Ap-p at each of the circumferential positions P1, P2, and P3 in this case.

As a cuff of Comparative Example 1, the present inventors prepared a cuff in which a sound acquisition fluid bag 22 was disposed under a pressing fluid bag 23 in the same manner as in the conventional example. The cuff of Comparative Example 1 is configured similarly to the cuff 20 except for that point. FIG. 7C illustrates an amplitude Ap-p (unit; volt) of a peak-to-peak of the K sound signal Ks in a case where the circumferential position of the sound acquisition fluid bag 22 of the cuff of Comparative Example 1 is changed in three ways as indicated by P1, P2, and P3. In this case, an average value of the amplitudes Ap-p at the circumferential position P1 was about 0.73 volts, an average value of the amplitudes Ap-p at the circumferential position P2 was about 1.28 volts, and an average value of the amplitudes Ap-p at the circumferential position P3 was about 0.92 volts. As a result, a change amount (maximum difference) Dv2 of the average value of the amplitudes Ap-p due to the change of the circumferential positions P1, P2, and P3 was about 0.55 volt. Note that dv1′, dv2′, and dv3′ indicate a variation range of the amplitude Ap-p at the individual circumferential positions P1, P2, and P3 in this case.

As can be seen by comparing the results of FIGS. 7B and 7C, the former change amount Dv1 is smaller than the latter change amount (maximum difference) Dv2. That is, in the cuff 20, even if the position (in particular, the circumferential position) where the cuff 20 (pressing fluid bag 23) is worn with respect to the site 90 to be measured varies, the influence on the level of the sound entering the pressing fluid bag 23 from the artery 91 passing through the site 90 to be measured is smaller than that in the conventional example. As a result, in the cuff 20, the sound collection by the sound acquisition fluid bag 22 is stabilized, and thus the K sound signal Ks representing the Korotkoff sound can be stably acquired.

As described above, with the configuration in which the sound acquisition fluid bag 22 is disposed on the pressing fluid bag 23, it is possible to verify the effect that the Korotkoff sound can be stably acquired even if the position (in particular, the circumferential position) where the cuff 20 is worn with respect to the site 90 to be measured varies.

### (Verification Experiment 2)

In order to verify the effect that the pulse sound (pulse wave sound) can be prevented from being mixed from the first fluid system FS1 with respect to the sound (including the Korotkoff sound component) passing through the second fluid system FS2 by the configuration in which the first fluid system FS1 and the second fluid system FS2 are separated from each other so as not to be capable of flowing a fluid, the present inventors conducted the following verification experiment.

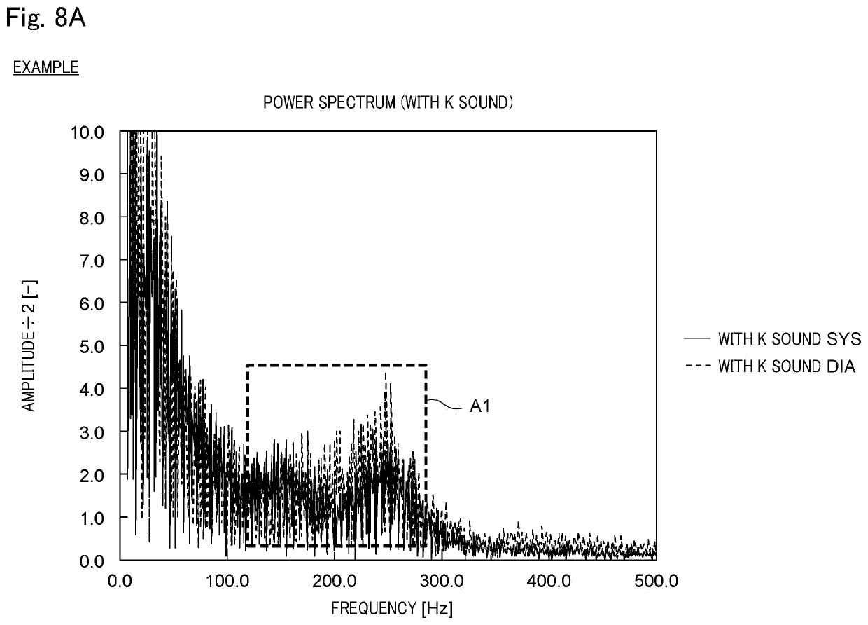

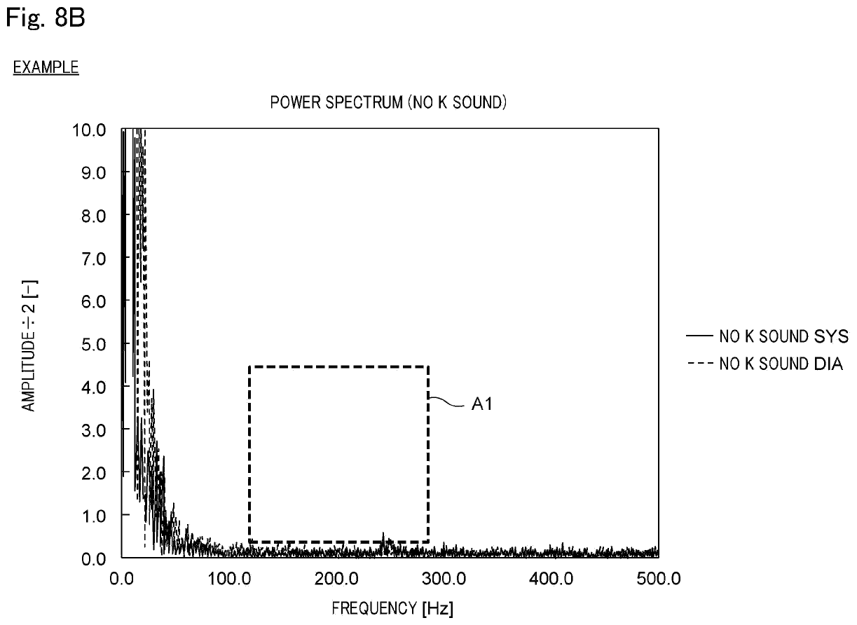

FIG. 8A illustrates a power spectrum of a sound acquired by the microphone 35 in a case where the cuff 20 acquires a sound from the site 90 to be measured (with K sound). FIG. 8B illustrates a power spectrum of a sound acquired by the microphone 35 in a case where the cuff 20 does not acquire a sound from the site 90 to be measured (no K sound). It can be seen that the spectrum of the Korotkoff sound appears in a range A1 of about 120 Hz to 300 Hz in FIG. 8A, whereas the spectrum of the Korotkoff sound does not appear in a range A1 in FIG. 8B. As described above, in the sphygmomanometer 100 including the cuff 20, the K sound signal Ks representing the Korotkoff sound can be reliably acquired.

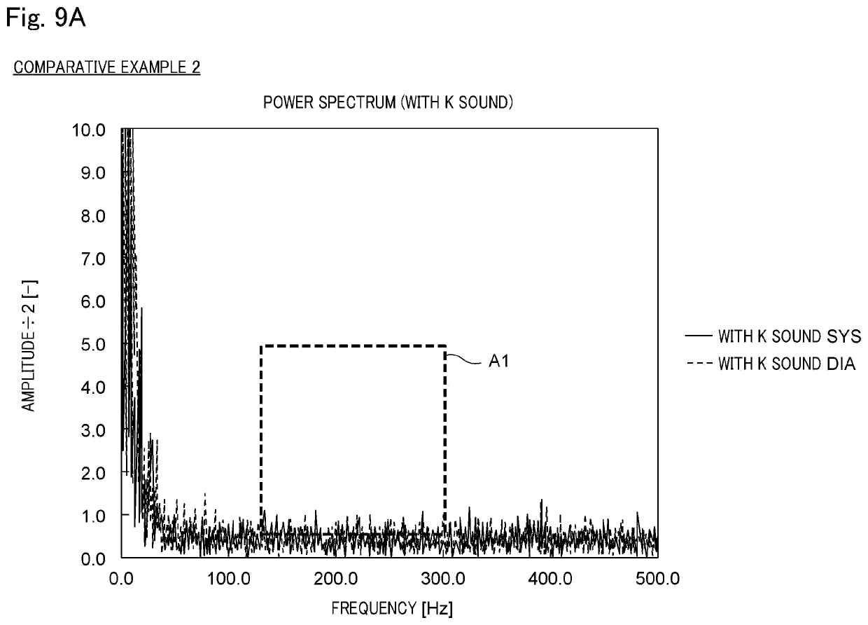

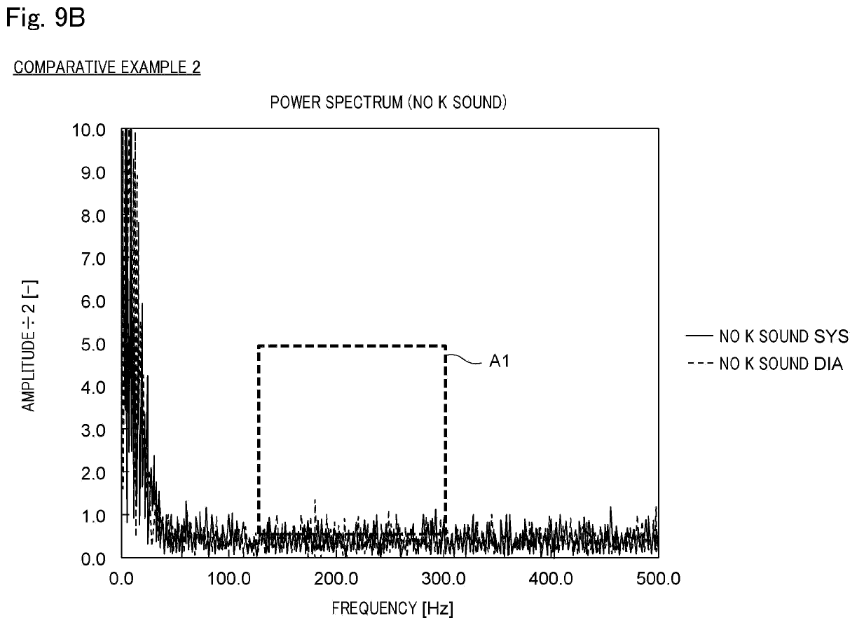

The present inventors prepared a cuff of Comparative Example 2 in which air pipes 37 and 38 were a common air pipe. The cuff of Comparative Example 2 is configured similarly to the cuff 20 except for that point. FIG. 9A illustrates a power spectrum of a sound acquired by a microphone 35 when the cuff of Comparative Example 2 acquires a sound from a site 90 to be measured (with K sound). FIG. 9B illustrates a power spectrum of a sound acquired by the microphone when the cuff of Comparative Example 2 does not acquire a sound from the site 90 to be measured (no K sound). In both FIGS. 9A and 9B, it can be seen that the spectrum of the Korotkoff sound does not appear within a range A1 of about 120 Hz to 300 Hz. The reason for this is considered to be that in FIG. 9A, the spectrum of the Korotkoff sound is buried in the background noise (including the component of the pulse sound). Note that in FIGS. 8A, 8B, 9A, and 9B, the maximum value in the acquired spectrum data is normalized to 10.

From these results, with the configuration in which the first fluid system FS1 and the second fluid system FS2 are separated from each other so as not to be capable of flowing a fluid, it is possible to verify the effect that the pulse sound (pulse wave sound) can be prevented from being mixed from the first fluid system FS1 with respect to the sound (including the Korotkoff sound component) passing through the second fluid system FS2. Note that it can be said that this effect can be obtained even if the sound acquisition fluid bag 22 is disposed below the pressing fluid bag 23.

### (Verification Experiment 3)

In order to verify the effect that an appropriate amount of air can be sealed in the sound acquisition fluid bag 22 to acquire the Korotkoff sound from the site 90 to be measured via the pressing fluid bag 23 by closing the atmosphere release valve 34 (step S4 in FIG. 5) at a stage before starting pressurization of the pressing fluid bag 23 after the cuff 20 is worn on the site 90 to be measured at the time of the blood pressure measurement, the present inventors conducted the following verification experiment.

As described above, FIG. 11 illustrates the K sound signal Ks representing the acquired Korotkoff sound in the blood pressure measurement flow described above, that is, in a case where the atmosphere release valve 34 is closed (step S4 in FIG. 5) before the pressurization of the pressing fluid bag 23 is started after the cuff 20 is worn on the site 90 to be measured. In the example of FIG. 11, the K sound signal Ks starts to be observed at time t2, gradually increases to indicate a maximum value, then gradually decreases, and immediately disappears at time t3.

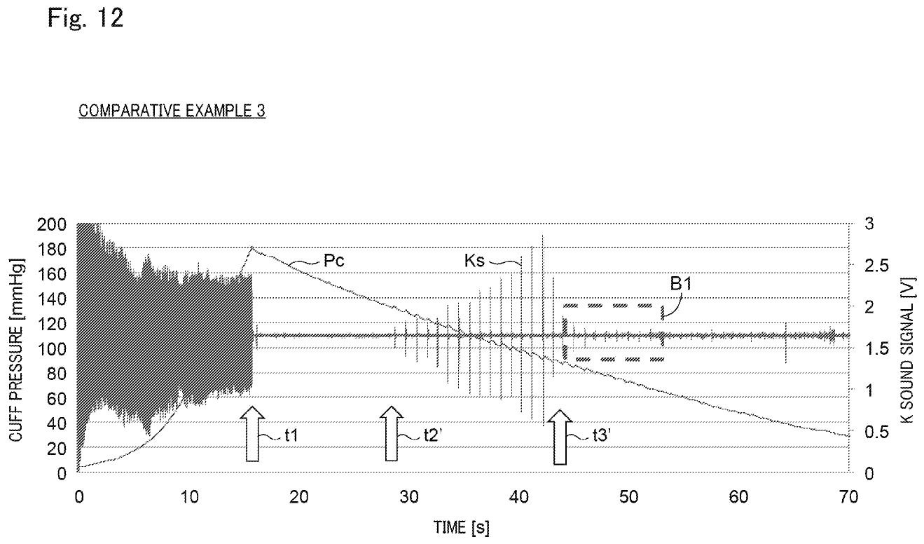

On the other hand, FIG. 12 illustrates a K sound signal Ks representing the acquired Korotkoff sound in a case where the atmosphere release valve 34 is closed before the cuff 20 is worn on the site 90 to be measured (Comparative Example 3), that is, in a case where air exceeding the appropriate amount remains in the sound acquisition fluid bag 22. In this case, the K sound signal Ks starts to be observed at time t2′ corresponding to the systolic blood pressure SYS, gradually increases to show a maximum value, and then gradually decreases, but does not disappear immediately after time t3′ corresponding to the diastolic blood pressure DIA, and slowly disappears as illustrated in a region B1 indicated by a broken line. The reason for this is considered to be that the frequency component at the time of the diastolic blood pressure DIA is lower than the frequency component at the time of the systolic blood pressure SYS, and thus is easily affected by the pulse wave (vibration). In this manner, in a case where the K sound signal Ks remains after time t3′, it is difficult to determine at which time the cuff pressure corresponds to the diastolic blood pressure DIA.

As a result, at the time of blood pressure measurement, by closing the atmosphere release valve 34 at a stage before starting pressurization of the pressing fluid bag 23 after the cuff 20 is worn on the site 90 to be measured (step S4 in FIG. 5), it is possible to verify the effect that an appropriate amount of air can be sealed in the sound acquisition fluid bag 22 in order to acquire the Korotkoff sound from the site 90 to be measured via the pressing fluid bag 23.

### (Modification 1)

In the cuff 20, as described with reference to FIGS. 3A and 3B, the sound acquisition fluid bag 22 and the pressing fluid bag 23 are configured by the four sheets 22a, 22b, 23a, and 23b. However, the present invention is not limited thereto.

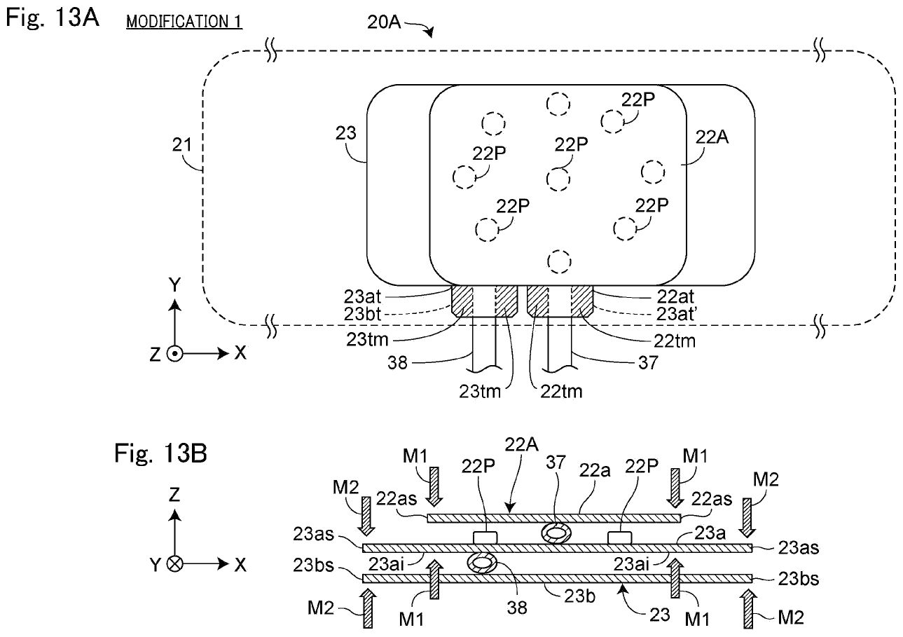

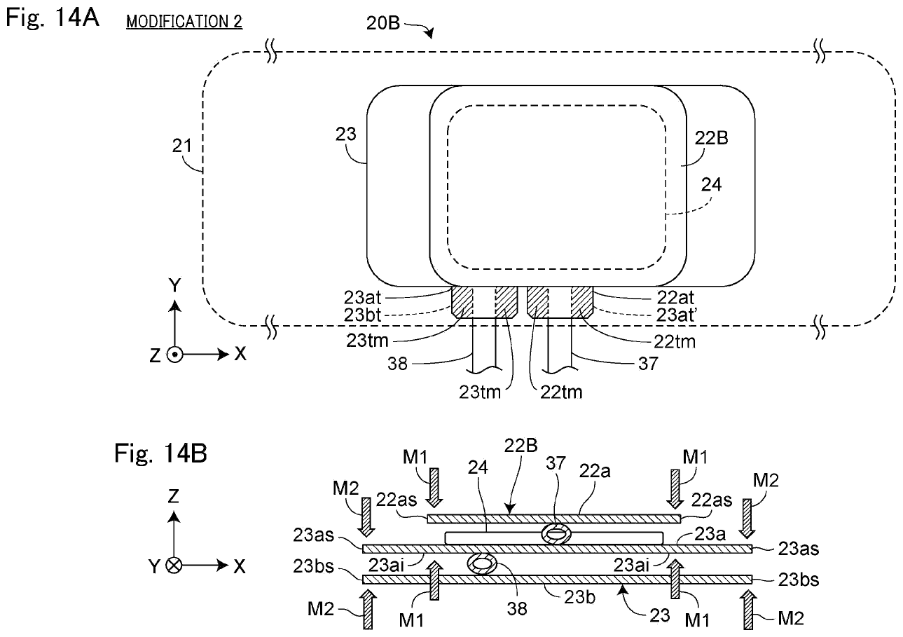

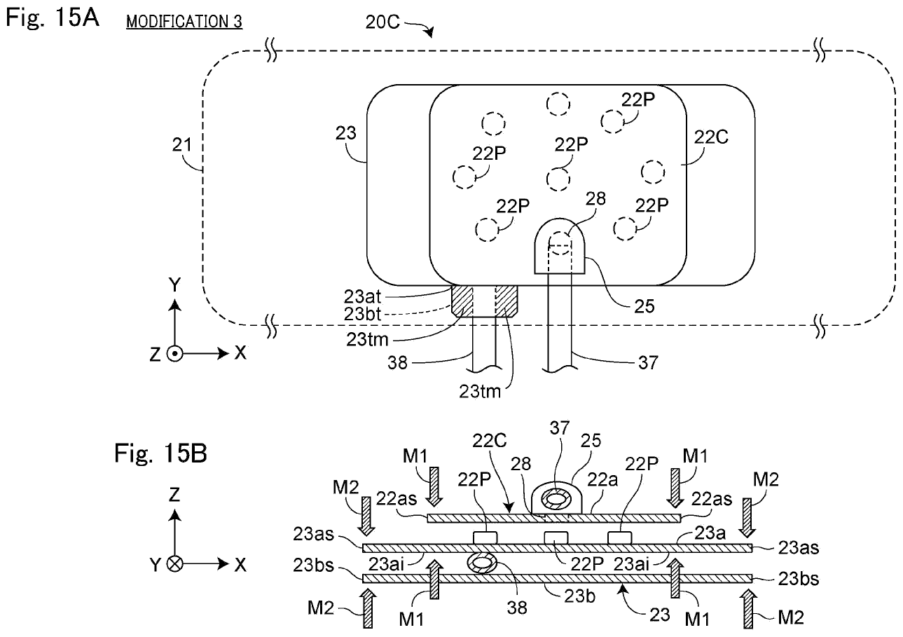

FIGS. 13A and 13B illustrate, as a cuff 20A of Modification 1 obtained by modifying the cuff 20, an example in which the sound acquisition fluid bag 22 and the pressing fluid bag 23 are configured by three sheets 22a, 23a, and 23b in correspondence with FIGS. 3A and 3B. Note that the same components as those in FIGS. 3A and 3B are denoted by the same reference signs, and redundant description will be appropriately omitted (The same applies to FIGS. 14A, 14B, 15A, and 15B described later). Furthermore, in FIG. 13B, illustration of the outer cloth 21 and the inner cloth 29 is omitted for simplicity (The same applies to FIGS. 14B, and 15B described later).

In the cuff 20A, as can be seen from FIG. 13B, in a sound acquisition fluid bag 22A, a peripheral edge part 22as of the sheet 22a and a part 23ai corresponding to the peripheral edge part 22as of the upper sheet 23a (sheet on a side of the sound acquisition fluid bag 22) forming the pressing fluid bag 23 are annularly joined (in this example, welded) to each other as indicated by arrows M1 to form a bag shape. As in the cuff 20, the pressing fluid bag 23 is formed in a bag shape by annularly joining (in this example, welding) the peripheral edge parts 23as and 23bs of the pair of sheets 23a and 23b to each other as indicated by arrows M2. That is, among the pair of sheets 22a and 22b of the sound acquisition fluid bag 22 illustrated in FIG. 3B, the sheet 22b on the side of the pressing fluid bag 23 is common to the upper sheet 23a among the pair of sheets 23a and 23b of the pressing fluid bag 23, and is omitted. As described above, in the cuff 20A, since the sound acquisition fluid bag 22 and the pressing fluid bag 23 are configured by the three sheets 22a, 23a, and 23b, the structure is simplified. Note that joining indicated by arrows M1 is performed first, and then joining indicated by arrows M2 is performed.

Note that in the cuff 20A, as can be seen from FIG. 13A, the upper sheet 23a constituting the pressing fluid bag 23 has a tab 23at′ at a position corresponding to a tab 22at of the sound acquisition fluid bag 22A, in addition to a tab 23at. In a state where the air pipe 37 is sandwiched between the tabs 22at and 23at′, parts 22tm and 22tm (indicated by hatching) of the tabs 22at and 23at′ corresponding to both sides of an air pipe 37 are entirely welded, so that the air pipe 37 is connected to the sound acquisition fluid bag 22A so as to be capable of flowing a fluid.

Furthermore, in the cuff 20A, a plurality of protrusions 22p, 22p, . . . as spacers are integrally formed on the upper surface of the upper sheet 23a constituting the pressing fluid bag 23. This prevents the sheets 22a and 23a constituting the sound acquisition fluid bag 22A from coming into close contact with each other.

### (Modification 2)

In the cuffs 20 and 20A, the spacers in the sound acquisition fluid bags 22 and 22A are formed of the plurality of protrusions 22p, 22p, . . . integrally formed with the sheet 22b or 23a, respectively. However, the present invention is not limited thereto.

FIGS. 14A and 14B illustrate an example in which spacers are formed of a sponge sheet 24 as a cuff 20B of Modification 2 obtained by further modifying the cuff 20A of Modification 1, in correspondence with FIGS. 13A and 13B.

In the cuff 20B, as can be seen from FIG. 14B, the sponge sheet 24 as a spacer is provided in a gap between a pair of sheets 22a and 23a facing each other and forming a sound acquisition fluid bag 22B. As can be seen from FIG. 14A, the sponge sheet 24 has a rounded rectangular shape having a slightly smaller dimension than the upper sheet 22a forming the sound acquisition fluid bag 22B in a plane along an outer cloth 21. This is to secure a margin for a bonded part indicated by arrows M1 in FIG. 14B. The cuff 20B is configured similarly to the cuff 20A in other points.

The cuff 20B can be easily manufactured since it is not necessary to use a sheet in which the plurality of protrusions 22p, 22p, . . . are integrally formed.

The sponge sheet 24 may or may not be bonded to any one or both of the pair of sheets 22a and 23a constituting the sound acquisition fluid bag 22B.

### (Modification 3)

In the cuffs 20, 20A, and 20B, the air pipe 37 is connected to the sound acquisition fluid bags 22, 22A, and 22B using the tabs 22at and 22bt or 22at and 23at′, respectively. However, the present invention is not limited thereto.

FIGS. 15A and 15B illustrate, as a cuff 20C of Modification 3 obtained by further modifying the cuff 20A of Modification 1, an example in which an air pipe 37 is connected to a sound acquisition fluid bag 22C using a cap 25, in a manner corresponding to FIGS. 13A and 13B.

In the cuff 20C, the dome-shaped cap 25 is integrally attached to an upper surface of an upper sheet 22a forming the sound acquisition fluid bag 22C. A through hole 28 penetrating the sheet 22a in the thickness direction Z is provided in a part of the sheet 22a corresponding to the cap 25. In this example, an end part of the air pipe 37 is hermetically fitted into and attached to the cap 25.

The cuff 20C can be easily manufactured as compared with the cuffs 20, 20A, and 20B since it does not take time and effort to weld the tabs 22at and 22bt or 22at and 23at′.

In the above example, each of the pressing fluid bag 23 and the sound acquisition fluid bags 22, 20A, 20B, and 20C has a rounded rectangular shape in the plane along the outer cloth 21, but the present invention is not limited thereto. The planar shape thereof may be a round corner square, an elliptical shape, a circular shape, or the like.

Furthermore, in the above example, the site 90 to be measured is the upper arm (in particular, the left upper arm), but the present invention is not limited thereto. The site 90 to be measured may be a right upper arm, an upper limb other than the upper arm such as a wrist, or a lower limb such as an ankle.

As described above, a blood pressure measuring cuff that compresses a site to be measured to acquire a Korotkoff sound, the blood pressure measuring cuff of the present disclosure comprises:

an outer cloth extending in a longitudinal direction in a band shape and surrounding the site to be measured;

a pressing fluid bag that is provided to extend along the longitudinal direction on a side of the outer cloth facing the site to be measured, and compresses the site to be measured;

a sound acquisition fluid bag that is provided between the outer cloth and the pressing fluid bag in a thickness direction perpendicular to the outer cloth, and acquires a sound from the site to be measured via the pressing fluid bag;

a first fluid pipe connected to the pressing fluid bag so as to be capable of flowing a fluid; and

a second fluid pipe connected to the sound acquisition fluid bag so as to be capable of flowing a fluid, separately from the first fluid pipe.

In the present specification, the “site to be measured” includes an upper limb such as an upper arm and a wrist or a lower limb such as an ankle, and typically refers to a rod-like site.

The “side facing the site to be measured” means a side facing the site to be measured in a state where the blood pressure measuring cuff is worn around the site to be measured (This is referred to as a “worn state”).

In the blood pressure measuring cuff, the “longitudinal direction” means a direction in which the outer cloth extends in a band shape, and corresponds to a circumferential direction surrounding the site to be measured in the worn state. A “width direction” described later means a direction perpendicular to the longitudinal direction in a plane along the outer cloth, and corresponds to a direction in which an artery passes through the site to be measured in the worn state. Furthermore, the “thickness direction” means a direction perpendicular to both the longitudinal direction and the width direction (that is, the outer cloth), and corresponds to a direction perpendicular to an outer peripheral surface of the site to be measured in the worn state.

In the blood pressure measuring cuff of the present disclosure, the pressing fluid bag is connected to a pressure device (typically including pumps and valves) provided outside the blood pressure measuring cuff through the first fluid pipe so as to be capable of flowing a fluid. The sound acquisition fluid bag is connected to a sound detection device (typically including a microphone) provided outside the blood pressure measuring cuff through the second fluid pipe so as to be capable of flowing a fluid. The blood pressure measuring cuff is worn such that the longitudinal direction of the cuff surrounds the site to be measured. In this worn state, the pressing fluid bag, the sound acquisition fluid bag, and the outer cloth are arranged in this order with respect to the site to be measured in the thickness direction. In this worn state, at the time of blood pressure measurement, air is supplied to the pressing fluid bag through the first fluid pipe by the pressure device. As a result, the pressing fluid bag is pressurized. In this pressurization process, the expansion of the pressing fluid bag together with the sound acquisition fluid bag in a direction away from the site to be measured is regulated by the outer cloth as a whole. Therefore, the pressing fluid bag expands in a direction of pressing the site to be measured. As a result, the site to be measured is compressed, and the artery passing through the site to be measured is ischemic. Subsequently, air is gradually discharged from the pressing fluid bag through the first fluid pipe by the pressure device. As a result, the pressure of the pressing fluid bag is gradually reduced. For example, in this depressurization process, the sound acquisition fluid bag acquires a sound from the site to be measured via the pressing fluid bag. Moreover, the sound detection device detects the sound acquired by the sound acquisition fluid bag through the second fluid pipe. Then, the Korotkoff sound is extracted based on an output of the sound detection device according to the sound from the sound acquisition fluid bag, and a blood pressure of the site to be measured is measured.

As described above, in the blood pressure measuring cuff, the sound acquisition fluid bag acquires the sound from the site to be measured via the pressing fluid bag. In the worn state, the pressing fluid bag extends along the circumferential direction of the site to be measured. Therefore, even if a position (in particular, a circumferential position) where the cuff is worn with respect to the site to be measured varies, the influence on a level of the sound entering the pressing fluid bag from the artery passing through the site to be measured is small, and as a result, the sound collection by the sound acquisition fluid bag is stabilized as compared with the conventional example. Therefore, the Korotkoff sound can be stably acquired.

In the blood pressure measuring cuff of one embodiment, a first fluid system including the pressing fluid bag and the first fluid pipe, and a second fluid system including the sound acquisition fluid bag and the second fluid pipe are separated from each other so as not to be capable of flowing a fluid.

In the blood pressure measuring cuff of this embodiment, it is possible to prevent a pulse sound (pulse wave sound) from being mixed from the first fluid system with respect to the sound (including the. Korotkoff sound component) passing through the second fluid system. Therefore, the Korotkoff sound can be more stably acquired.

In the blood pressure measuring cuff of one embodiment, the sound acquisition fluid bag includes a pair of sheets facing each other in the thickness direction, and the pair of sheets are joined to each other to form a bag shape, and a spacer that prevents the pair of sheets from coming into close contact with each other is provided in a gap between the pair of sheets facing each other.

In the blood pressure measuring cuff of this embodiment, since the spacer is provided in the gap between the pair of sheets facing each other, the pair of sheets is prevented from coming into close contact with each other. Therefore, the sound acquisition fluid bag can stably acquire the sound from the site to be measured via the pressing fluid bag. As a result, the Korotkoff sound can be more stably acquired.

In the blood pressure measuring cuff of one embodiment, the spacer includes a protrusion integrally formed on the sheets.

In the blood pressure measuring cuff of this embodiment, the spacer can be easily configured.

In the blood pressure measuring cuff of one embodiment, the pressing fluid bag includes a pair of sheets facing each other in the thickness direction, and the pair of sheets are annularly joined to each other to form a bag shape, the sound acquisition fluid bag includes a pair of sheets facing each other in the thickness direction, and the pair of sheets are annularly joined to each other to form a bag shape, and the sheet on a side of the pressing fluid bag out of the pair of sheets of the sound acquisition fluid bag is common to the sheet on a side of the sound acquisition fluid bag out of the pair of sheets of the pressing fluid bag.

The term “joined” means that they are joined together by welding, adhesion, or the like.

In the blood pressure measuring cuff of this embodiment, the sheet on the side of the pressing fluid bag out of the pair of sheets of the sound acquisition fluid bag is common to the sheet on the side of the sound acquisition fluid bag out of the pair of sheets of the pressing fluid bag. Therefore, since the pressing fluid bag and the sound acquisition fluid bag are composed of three sheets, the structure is simplified.

In the blood pressure measuring cuff of one embodiment, the site to be measured is an upper arm, a dimension of the pressing fluid bag in the longitudinal direction is set within a range of 167 mm to 380 mm, and a dimension of the pressing fluid bag in a width direction perpendicular to the longitudinal direction in a plane along the outer cloth is set within a range of 90 mm to 180 mm, and a dimension of the sound acquisition fluid bag in the longitudinal direction is set within a range of 41.8 mm to 380 mm, and a dimension of the sound acquisition fluid bag in the width direction is set within a range of 45 mm to 180 mm.

The dimension in the longitudinal direction and the dimension in the width direction are collectively referred to as “plane-direction dimension” as appropriate.

In the blood pressure measuring cuff of this embodiment, it is possible to fit and wear the blood pressure measuring cuff to subjects of various arm circumferences due to the setting of the plane-direction dimension of the pressing fluid bag. Furthermore, even if a position (in particular, a circumferential position) where the cuff is worn on the upper arm as the site to be measured varies, the pressing fluid bag can stably face the artery passing through the upper arm. Furthermore, due to the setting of the plane-direction dimension of the sound acquisition fluid bag, in a case where the pair of sheets is made of, for example, a general polyurethane resin, the natural frequency of the sound acquisition fluid bag can be set to substantially the same order with respect to the main frequency component of the Korotkoff sound. Therefore, the sound acquisition fluid bag can efficiently acquire the Korotkoff sound component from the site to be measured.

In the blood pressure measuring cuff of one embodiment, the site to be measured is a wrist, a dimension of the pressing fluid bag in the longitudinal direction is set to 140 mm, and a dimension of the pressing fluid bag in a width direction perpendicular to the longitudinal direction in a plane along the outer cloth is set to 60 mm, and a dimension of the sound acquisition fluid bag in the longitudinal direction is set within a range of 35 mm to 140 mm, and a dimension of the sound acquisition fluid bag in the width direction is set within a range of 30 mm to 60 mm.

In the blood pressure measuring cuff of this embodiment, the sound acquisition fluid bag can efficiently acquire the Korotkoff sound component from the site to be measured.

In the blood pressure measuring cuff of one embodiment, the dimension of the sound acquisition fluid bag in the longitudinal direction is set to ½ of the dimension of the pressing fluid bag in the longitudinal direction, and the dimension of the sound acquisition fluid bag in the width direction is set to be identical with the dimension of the pressing fluid bag in the width direction.

In the blood pressure measuring cuff of this embodiment, the sound acquisition fluid bag can efficiently acquire the Korotkoff sound component from the site to be measured.

In another aspect, a sphygmomanometer that calculates a blood pressure by a Korotkoff sound generated by a site to be measured, the sphygmomanometer of the present disclosure comprises:

the blood pressure measuring cuff described above;

a pressure device that is connected to the first fluid pipe so as to be capable of flowing a fluid, and supplies a fluid to the pressing fluid bag through the first fluid pipe to pressurize the pressing fluid bag or discharges a fluid from the pressing fluid bag through the first fluid pipe to depressurize the pressing fluid bag;

a sound detection device that is connected to the second fluid pipe so as to be capable of flowing a fluid, and detects a sound from the sound acquisition fluid bag through the second fluid pipe;

a first fluid system including the pressing fluid bag and the first fluid pipe;

a second fluid system including the sound acquisition fluid bag and the second fluid pipe, the first fluid system and the second fluid system being maintained so as not to be capable of flowing a fluid each other; and a blood pressure calculation unit that opens and closes the atmosphere release valve as the pressure device pressurizes or depressurizes the pressing fluid bag, and calculates a blood pressure of the site to be measured based on an output of the sound detection device according to the sound from the sound acquisition fluid bag.

The “pressure device” typically includes a pump and a valve.

The “sound detection device” typically includes a microphone.

In the sphygmomanometer of the present disclosure, the blood pressure measuring cuff is worn so as to surround the site to be measured. In this worn state, at the time of blood pressure measurement, air is supplied to the pressing fluid bag through the first fluid pipe by the pressure device. As a result, the pressing fluid bag is pressurized. In this pressurization process, the expansion of the pressing fluid bag together with the sound acquisition fluid bag in a direction away from the site to be measured is regulated by the outer cloth as a whole. Therefore, the pressing fluid bag expands in a direction of pressing the site to be measured. As a result, the site to be measured is compressed, and the artery passing through the site to be measured is ischemic. Subsequently, air is gradually discharged from the pressing fluid bag through the first fluid pipe by the pressure device. As a result, the pressure of the pressing fluid bag is gradually reduced. For example, in this depressurization process, the sound acquisition fluid bag acquires a sound from the site to be measured via the pressing fluid bag. Moreover, the sound detection device detects the sound acquired by the sound acquisition fluid bag through the second fluid pipe. Then, the blood pressure calculation unit extracts the Korotkoff sound based on the output of the sound detection device according to the sound from the sound acquisition fluid bag, and calculates the blood pressure of the site to be measured.

In this sphygmomanometer, the Korotkoff sound can be stably acquired by the blood pressure measuring cuff, and thus the blood pressure can be accurately measured.

In the sphygmomanometer of one embodiment, an atmosphere release valve that is connected to the second fluid pipe so as to be capable of flowing a fluid, and is capable of closing the second fluid pipe or opening the second fluid pipe to atmospheric pressure, wherein the blood pressure calculation unit closes the atmosphere release valve to seal the second fluid system before the pressure device starts pressurizing the pressing fluid bag after the blood pressure measuring cuff is worn on the site to be measured.

In the sphygmomanometer according to this embodiment, the sound acquisition fluid bag can be maintained in a state where an appropriate amount of air is sealed during the pressurization process and the depressurization process by the blood pressure calculation unit. The sound acquisition fluid bag can efficiently acquire the Korotkoff sound component from the site to be measured. Therefore, the blood pressure can be measured more accurately.

As is clear from the above, according to the blood pressure measuring cuff of the present disclosure, the Korotkoff sound can be stably acquired. Furthermore, according to the sphygmomanometer of the present disclosure, the blood pressure can be accurately measured.

The above embodiments are illustrative, and various modifications can be made without departing from the scope of the present invention. It is to be noted that the various embodiments described above can be appreciated individually within each embodiment, but the embodiments can be combined together. It is also to be noted that the various features in different embodiments can be appreciated individually by its own, but the features in different embodiments can be combined.

1. A blood pressure measuring cuff that compresses a site to be measured to acquire a Korotkoff sound, the blood pressure measuring cuff comprising:
an outer cloth extending in a longitudinal direction in a band shape and surrounding the site to be measured;
a pressing fluid bag that is provided to extend along the longitudinal direction on a side of the outer cloth facing the site to be measured, and compresses the site to be measured;
a sound acquisition fluid bag that is provided between the outer cloth and the pressing fluid bag in a thickness direction perpendicular to the outer cloth, and acquires a sound from the site to be measured via the pressing fluid bag;
a first fluid pipe connected to the pressing fluid bag so as to be capable of flowing a fluid; and
a second fluid pipe connected to the sound acquisition fluid bag so as to be capable of flowing a fluid, separately from the first fluid pipe.

2. The blood pressure measuring cuff according to claim 1, wherein
a first fluid system including the pressing fluid bag and the first fluid pipe, and a second fluid system including the sound acquisition fluid bag and the second fluid pipe are separated from each other so as not to be capable of flowing a fluid.

3. The blood pressure measuring cuff according to claim 1, wherein
the sound acquisition fluid bag includes a pair of sheets facing each other in the thickness direction, and the pair of sheets are joined to each other to form a bag shape, and
a spacer that prevents the pair of sheets from coming into close contact with each other is provided in a gap between the pair of sheets facing each other.

4. The blood pressure measuring cuff according to claim 3, wherein
the spacer includes a protrusion integrally formed on the sheets.

5. The blood pressure measuring cuff according to claim 1, wherein
the pressing fluid bag includes a pair of sheets facing each other in the thickness direction, and the pair of sheets are annularly joined to each other to form a bag shape,
the sound acquisition fluid bag includes a pair of sheets facing each other in the thickness direction, and the pair of sheets are annularly joined to each other to form a bag shape, and
the sheet on a side of the pressing fluid bag out of the pair of sheets of the sound acquisition fluid bag is common to the sheet on a side of the sound acquisition fluid bag out of the pair of sheets of the pressing fluid bag.

6. The blood pressure measuring cuff according to claim 1, wherein
the site to be measured is an upper arm,
a dimension of the pressing fluid bag in the longitudinal direction is set within a range of 167 mm to 380 mm, and a dimension of the pressing fluid bag in a width direction perpendicular to the longitudinal direction in a plane along the outer cloth is set within a range of 90 mm to 180 mm, and
a dimension of the sound acquisition fluid bag in the longitudinal direction is set within a range of 41.8 mm to 380 mm, and a dimension of the sound acquisition fluid bag in the width direction is set within a range of 45 mm to 180 mm.

7. The blood pressure measuring cuff according to claim 1, wherein
the site to be measured is a wrist,
a dimension of the pressing fluid bag in the longitudinal direction is set to 140 mm, and a dimension of the pressing fluid bag in a width direction perpendicular to the longitudinal direction in a plane along the outer cloth is set to 60 mm, and
a dimension of the sound acquisition fluid bag in the longitudinal direction is set within a range of 35 mm to 140 mm, and a dimension of the sound acquisition fluid bag in the width direction is set within a range of 30 mm to 60 mm.

8. The blood pressure measuring cuff according to claim 6, wherein
the dimension of the sound acquisition fluid bag in the longitudinal direction is set to ½ of the dimension of the pressing fluid bag in the longitudinal direction, and
the dimension of the sound acquisition fluid bag in the width direction is set to be identical with the dimension of the pressing fluid bag in the width direction.

9. A sphygmomanometer that calculates a blood pressure by a Korotkoff sound generated by a site to be measured, the sphygmomanometer comprising:
the blood pressure measuring cuff according to claim 1;
a pressure device that is connected to the first fluid pipe so as to be capable of flowing a fluid, and supplies a fluid to the pressing fluid bag through the first fluid pipe to pressurize the pressing fluid bag or discharges a fluid from the pressing fluid bag through the first fluid pipe to depressurize the pressing fluid bag;
a sound detection device that is connected to the second fluid pipe so as to be capable of flowing a fluid, and detects a sound from the sound acquisition fluid bag through the second fluid pipe;
a first fluid system including the pressing fluid bag and the first fluid pipe;
a second fluid system including the sound acquisition fluid bag and the second fluid pipe, the first fluid system and the second fluid system being maintained so as not to be capable of flowing a fluid each other; and
a blood pressure calculation unit that opens and closes the atmosphere release valve as the pressure device pressurizes or depressurizes the pressing fluid bag, and calculates a blood pressure of the site to be measured based on an output of the sound detection device according to the sound from the sound acquisition fluid bag.

10. The sphygmomanometer according to claim 9, further comprising
an atmosphere release valve that is connected to the second fluid pipe so as to be capable of flowing a fluid, and is capable of closing the second fluid pipe or opening the second fluid pipe to atmospheric pressure,
wherein the blood pressure calculation unit closes the atmosphere release valve to seal the second fluid system before the pressure device starts pressurizing the pressing fluid bag after the blood pressure measuring cuff is worn on the site to be measured.
